(19) United States
(12) Patent Application Publication
(10) Pub. No.: US 2005/0096697 A1
(43) Pub. Date: May 5, 2005

(54) Vascular insertion sheath with stiffened tip

(76) Inventors: Andrew Thomas Forsberg, Minneapolis, MN (US); Loran Paprocki, St. Louis Park, MN (US); William Fiehler, Maple Grove, MN (US); Russ Terwey, Brooklyn Center, MN (US)

Correspondence Address: L. Grant Foster;HOLLAND & HART LLP, 555 - 17th Street, Suite 3200, P.O. Box 8749, Denver, CO, 80201, US

(21) Appl. No.: 10/700,767

(22) Filed: Nov. 4, 2003

(51) Int. Cl. A61D 1/00; A61B 17/08

(52) U.S. Cl. 606/213

(57) ABSTRACT

The present invention provides a method and apparatus for sealing a subcutaneous tissue puncture. The method and apparatus reduce the occurrence of anchor shuttling by stiffening a tip or end portion of an insertion sheath that acts as a one-way valve to a closure device anchor. The stiffening of the tip reduces shuttling by reducing or eliminating the tendency of prior insertion sheath tips from puckering and creating a gap into which the anchor may reenter. The method of stiffening may take on many different mechanisms, several of which are disclosed.

### FIELD OF THE INVENTION

The present invention relates to medical devices, and, more particularly, to a vascular puncture sealing apparatus with features to aid in anchor function.

### BACKGROUND OF THE INVENTION

Various medical procedures, particularly cardiology procedures, involve accessing a corporeal vessel or other lumen through a percutaneous sheath. Insertion of the sheath necessarily requires a hole or opening in the vessel wall so that a medical procedure can be performed through the sheath. After the particular medical procedure has been performed, the sheath must eventually be removed from the vessel and the access hole in the vessel wall must be closed.

A number of prior vascular closure devices have been developed to close the hole or puncture in the vessel wall. Closing the hole typically involves packing a resorbable sealing plug at the hole or sandwiching the hole between the sealing plug and an anchor. Examples of prior vascular closure devices are described in U.S. Pat. Nos. 6,179,863; 6,090,130; and 6,045,569 and related patents that are hereby incorporated by reference.

Placing the sealing plug often comprises several steps. First, a puncture site is located. A puncture locator is placed in and through the insertion sheath such that an inlet port in the puncture locator resides outside a distal end of the insertion sheath a predetermined distance. The insertion sheath and puncture locator are inserted through the puncture into a blood vessel. As the distal end of the puncture locator penetrates the blood vessel, blood flows into the inlet port and out of a drip hole via a flow path through the puncture locator.

Blood exiting the drip hole indicates that the puncture locator and insertion sheath have just penetrated the blood vessel. To ensure proper placement of the insertion sheath and subsequently the closure device, the insertion sheath and puncture locator are normally backed out of the vessel until blood stops flowing from the drip hole. Next, the insertion sheath and puncture locator are re-inserted into the blood vessel until blood starts flowing again from the drip hole. Proper depth of penetration and location of the assembly is established by continuing to insert an additional predetermined distance, for example, an operator often inserts the assembly 1 to 2 centimeters further if the blood vessel is a femoral artery. After the insertion sheath is properly located, the puncture locator is removed and the vascular closure device is inserted through the sheath and into to the blood vessel.

After the vascular closure device is located in the blood vessel, an anchor at the distal end of the vascular closure device is usually deployed within the vessel. The anchor is initially aligned with a longitudinal axis of the closure device in the sheath. Inserting the anchor out of the distal end of the insertion sheath usually deploys the anchor, allowing it to rotate and align itself with an interior wall of the blood vessel. However, sometimes when the anchor is deployed, it may reenter the sheath instead of rotating and aligning with the blood vessel. This phenomenon is termed “shuttling.” Shuttling disables the function, and negates the benefit, of the device. Therefore, it is desirable to have an apparatus that reduces or eliminates anchor shuttle so that the closure device will function as expected. A failure with the closure device may introduce complications to the closure of the puncture.

One of the causes of shuttling is “pucker,” or the tendency of an insertion sheath tip to not seal against the closure device as it passes therethrough. If the insertion sheath tip puckers, a gap is formed between the closure device and the insertion sheath, and the anchor may reenter the insertion sheath via the gap. Therefore, it is desirable to have an apparatus reducing the tendency of insertion sheath “pucker” and therefore reduce the occurrence of anchor shuttle so that the closure device is most likely to succeed.

### SUMMARY OF THE INVENTION

In one of many possible embodiments, the present invention provides a tissue puncture closure assembly comprising a tissue puncture closure device having a distal and a proximal end, a vascular insertion sheath having a distal and a proximal end, where the distal end of the insertion sheath includes a tip portion stiffer than the remainder of the insertion sheath. The tip portion may have a concave fold and may include no more than half of a circumference of the insertion sheath. The tip portion may be stiffened by increasing the wall thickness of the tip portion to something greater than the wall thickness of the remainder of the insertion sheath. Adding a second layer of material or a stiffening ridge may also stiffen the tip portion. In addition, the tip portion may be corrugated or stiffened in other manners. The closure device may include a filament extending from the proximal end of the closure device to the distal end of the closure device, an anchor for insertion through a tissue wall puncture attached to the filament at the distal end of the closure device; and a sealing plug slidingly disposed about the filament at the distal end of the closure device for sealing the puncture.

Another embodiment provides a vascular insertion sheath including a flexible tubular member having a longitudinal axis, a distal end, and a proximal end; a hemostatic valve coupled to the proximal end of the tubular member, and a fold at the distal end of the tubular member. The fold comprises a higher stiffness coefficient than the tubular member. The higher stiffness coefficient may be provided by the addition of a layer of material over the fold, which may be added only at an edge of the fold. A fold having a thicker wall than the flexible tubular member may also provide the higher stiffness coefficient for the fold. In addition, a stiffening ridge or a corrugation may stiffen the fold.

The invention also provides a method of reducing anchor shuttle in a tissue puncture closure assembly, comprising stiffening a tip portion of an insertion sheath receptive of a tissue puncture closure device.

According to another embodiment the invention provides a method of making a vascular insertion sheath, comprising providing a flexible tubular member, tapering an end portion of the flexible tubular member, folding a section of the end portion into a concave depression, and stiffening at least a portion of the concave depression. The tapering may include inserting the flexible tubular member into a heated die and reforming the end portion. The folding may include inserting the flexible tubular member into a heated die and reforming the end portion. The stiffening may include inserting the flexible tubular member into a heated die and reforming at least part of the end portion into a thicker wall or a corrugated section. The stiffening may include applying a layer of material to at least part of the end portion, adding a ridge across the end portion in a direction transverse to a longitudinal axis of the flexible tubular member, or some other stiffening method.

The present invention also provides a tissue puncture closure assembly comprising a closure device for partial insertion into and sealing of an internal tissue wall puncture. The closure device includes a carrier tube having an anchor nest at a distal end, a filament extending through the carrier tube, an anchor attached to the filament at the distal end of the carrier tube and seated in the anchor nest, and an insertion sheath receptive of the carrier tube of the closure device. The insertion sheath includes a flexible tubular member having a longitudinal axis, a distal end, and a proximal end; a hemostatic valve coupled to the proximal end of the tubular member, and a fold at the distal end of the tubular member. The fold has a higher stiffness coefficient than the tubular member.

There is also provided a method of sealing a tissue puncture in an internal tissue wall accessible through a percutaneous incision, comprising providing a tissue puncture closure device having a carrier tube with a filament extending therethrough. The filament is connected at a distal end of the carrier tube to an anchor, and the anchor is initially seated in a nest disposed in the carrier tube. The filament is also connected to a sealing plug located proximal of the anchor for disposition and anchoring about the tissue puncture. The method includes inserting the tissue puncture closure device through an insertion sheath having a stiffened tip portion into the percutaneous incision, deploying the anchor into the tissue puncture, withdrawing the closure device from the percutaneous incision, and tamping the sealing plug toward the anchor.

The foregoing and other features, utilities and advantages of the invention will be apparent from the following more particular description of preferred embodiments of the invention as illustrated in the accompanying drawings.

### BRIEF DESCRIPTION OF THE DRAWINGS

The above and other objects and advantages of the present invention will be apparent upon consideration of the following detailed description, taken in conjunction with the accompanying drawings, in which like reference characters refer to similar, but not necessarily identical parts throughout, and in which:

FIG. 1A is a cut-away assembly view of a tissue puncture closure device and insertion sheath according to the prior art.

FIG. 1B is a detail of the cut-away section of FIG. 1A.

FIG. 2A is side view of the tissue puncture closure device of FIG. 1A engaged with the insertion sheath in a first position according to the prior art.

FIG. 2B is a detailed cross-sectional view of the tissue puncture closure device and insertion sheath of FIG. 2A.

FIG. 3 is a perspective view of a tip portion of the insertion sheath of FIG. 2A.

FIG. 4 is a front view of the insertion sheath of FIG. 3.

FIG. 5 is a cross-sectional view of the tissue puncture closure device of FIG. 1A in relation to the insertion sheath of FIG. 2A in a second position according to the prior art.

FIG. 6 is a cross-sectional view of the tissue puncture closure devices of FIG. 1A in relation to the insertion sheath of FIG. 2A in a third position according to the prior art.

FIG. 7 is a perspective view of the tissue puncture closure device of FIG. 1A and insertion sheath of FIG. 2A shown in relation to a patient with an anchor deployed according to the prior art.

FIG. 8A is a cross-sectional view of the tissue puncture closure device of FIG. 1A in relation to the insertion sheath of FIG. 3 in a fourth position illustrating shuttling according to the prior art.

FIG. 8B is a front view of the insertion sheath of FIG. 3 with the tissue puncture closure device of FIG. 1A inside of the insertion sheath.

FIG. 9 is a front cross-sectional view of a stiffened insertion sheath according to one embodiment of the present invention with a tip portion having a thickened wall.

FIG. 10 is a front view of a stiffened insertion sheath according to another embodiment of the present invention with a thickened edge tip portion.

FIG. 11A is a perspective view of a stiffened insertion sheath according to another embodiment of the present invention with stiffening ridges at the tip portion.

FIG. 11B is a cross-sectional side view of the stiffened insertion sheath of FIG. 1A.

FIG. 12A is a perspective view of a stiffened insertion sheath according to another embodiment of the present invention with a second layer added to the tip portion.

FIG. 12B is a cross-sectional side view of the stiffened insertion sheath of FIG. 12A.

FIG. 13A is a perspective view of a stiffened insertion sheath according to another embodiment of the present invention with a corrugated section of the tip portion.

FIG. 13B is a cross-sectional side view of the stiffened insertion sheath of FIG. 13A.

FIG. 14 is a perspective view of the tissue closure device of FIG. 1A in relation to a stiffened insertion sheath with an operator tamping a sealing plug according to the present invention.

### DETAILED DESCRIPTION

As mentioned above, vascular procedures are commonly performed throughout the world and require access to a lumen through a puncture. Most often, the lumen is a femoral artery. To close the puncture, many times a closure device is used to sandwich the puncture between an anchor and a sealing plug. However, there exists a possibility for the anchor to not deploy, disabling the function and negating the benefit of the device. The present invention describes methods and apparatus to reduce or eliminate non-deployment or “shuttle” of a closure device anchor. While the vascular instruments shown and described below include embodiments of particular insertion sheaths and puncture sealing devices, the application of principles described herein to reduce anchor shuttle is not limited to the specific devices shown. The principles described herein may be used to reduce anchor shuttle for any vascular closure assembly. Therefore, while the description below is directed primarily to arterial procedures and certain embodiments of a vascular closure assembly, the methods and apparatus are only limited by the appended claims.

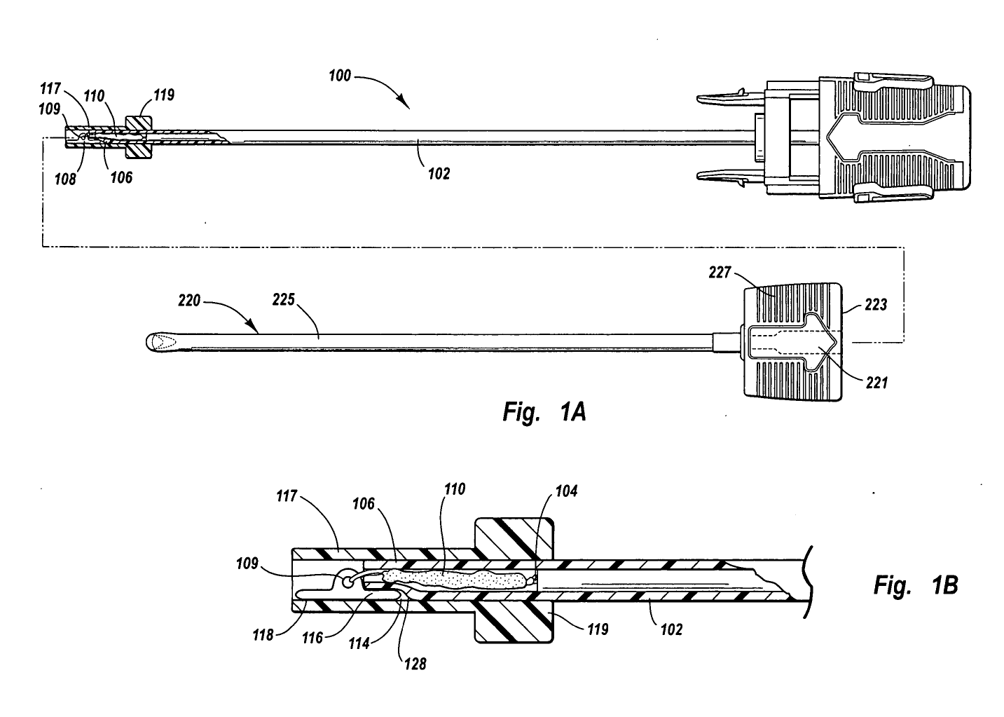

Referring now to the drawings, and in particular to FIGS. 1A-1B, a vascular puncture closure assembly including a closure device 100 and an insertion sheath 220 is shown according to the prior art. The vascular puncture closure device 100 includes a carrier tube 102 with a filament or suture 104 extending at least partially therethrough. External to the first or distal end 106 of the carrier tube is an anchor 108. The anchor is an elongated, stiff, low profile member with a protruding dome 109. The anchor 108 is typically made of a non-hemostatic biologically resorbable polymer.

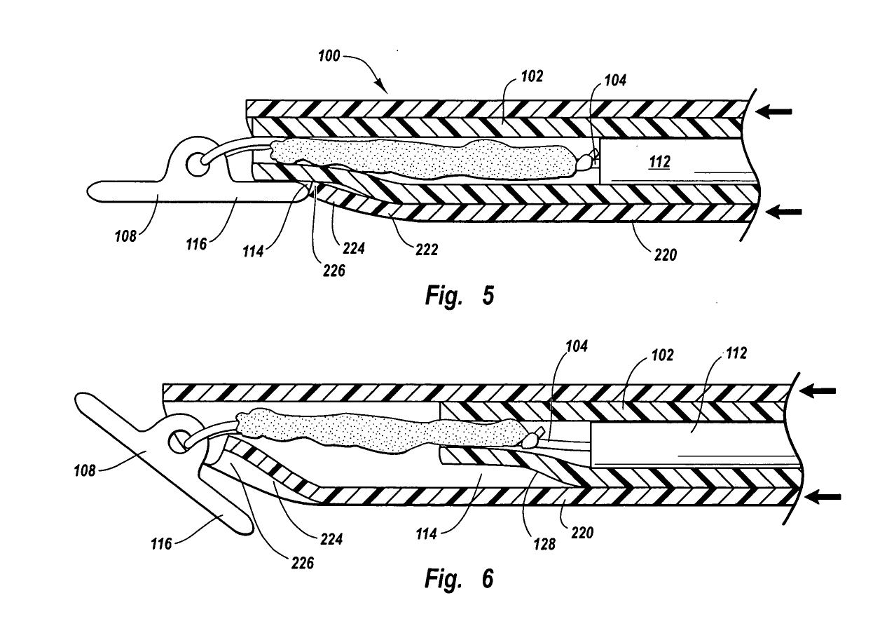

The suture 104 is also made of a biologically resorbable material and is threaded through the anchor 108 and back to a collagen sponge 110. The collagen sponge 110 is slidingly attached to the suture 104 as the suture passes distally through the carrier tube 102. However, as the suture traverses the anchor 108 and reenters the carrier tube 102, it is securely slip knotted proximal to the collagen sponge 110 to facilitate cinching of the collagen sponge 110 when the closure device 100 is properly placed and the anchor 108 deployed (see FIG. 5).

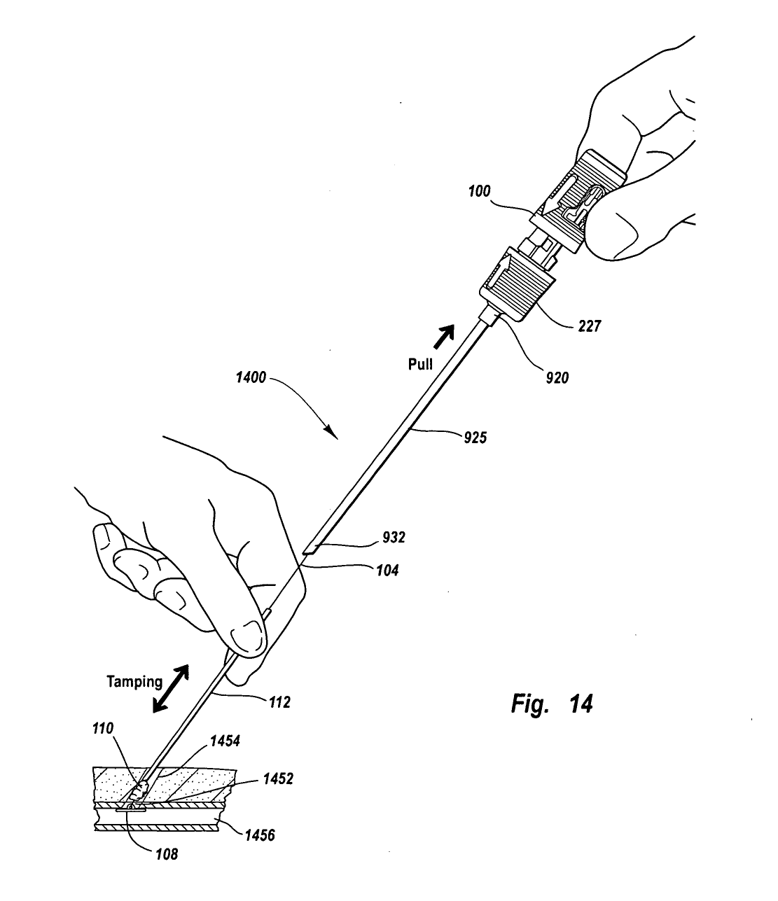

A tamping tube 112 is disposed in the carrier tube 102 proximal to the collagen sponge 110. The tamping tube 112 is slidingly mounted on the suture 104 and may be used by an operator to tamp the collagen sponge 110 toward the anchor 108 at an appropriate time to plug a percutaneous tissue puncture (See FIG. 14).

At the distal end 106 of the carrier tube 102 is a nest 114. Prior to deployment of the anchor 108 within an artery, the protruding dome 109 seats outside the distal end 106 of the carrier tube 102, and one end 116 of the anchor 108 rests in the nest 114. The nest 114 is typically crushed to a depth such that a surface 118 of the anchor 108 is flush with the outer diameter of the carrier tube 102. The nest 114 is crushed to a length that is longer than the end 116 of the anchor 108. The anchor 108 may be temporarily held in place in the nest 114 by a bypass tube 117 disposed over the distal end 106 of the carrier tube 102.

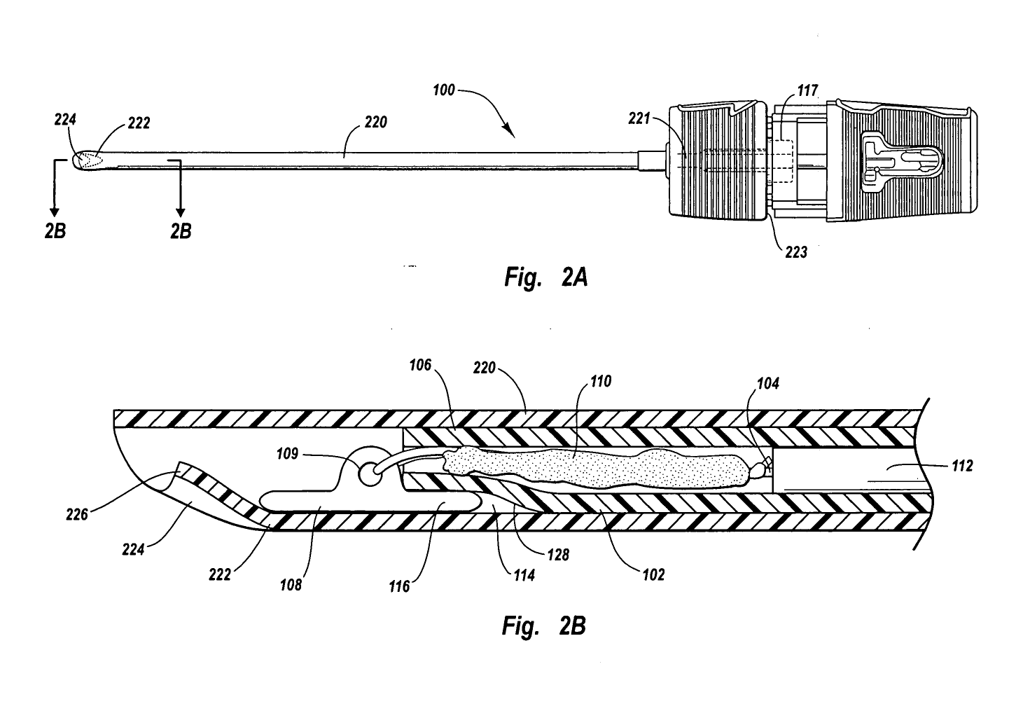

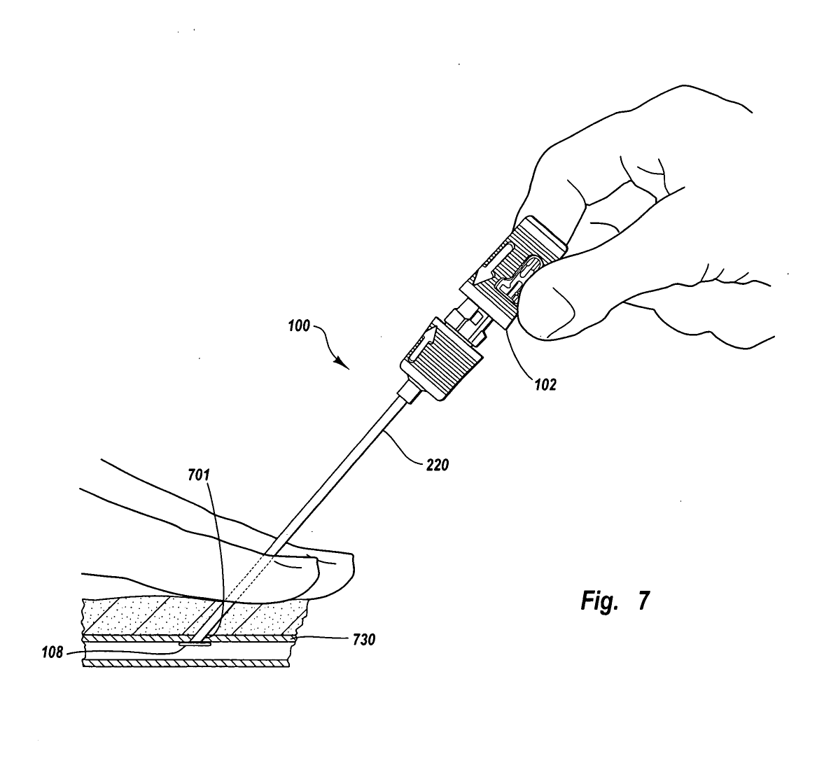

The flush arrangement of the anchor 108 and carrier tube 102 allows the anchor to be inserted into an insertion sheath 220 as shown in FIG. 2A-2B, and eventually through an arterial puncture 701 (shown in FIG. 7). However, the bypass tube 117 includes an oversized head 119 that prevents the bypass tube 117 from passing through an internal passage 221 of the insertion sheath 220. Therefore, as the puncture closure device 100 is inserted into the internal passage 221 of the insertion sheath 220, the oversized head 117 bears against a surface 223 of the insertion sheath 220. Further insertion of the puncture closure device 100 results in sliding movement between the carrier tube 102 and the bypass tube 116, releasing the anchor 108 from the bypass tube 116. However, the anchor 108 remains in the nest 114 following release from the bypass tube 116, limited in movement by the insertion sheath 220.

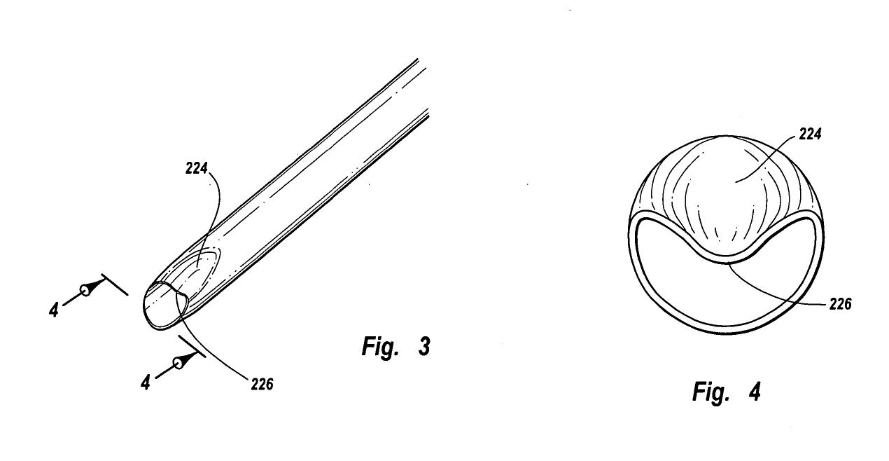

The insertion sheath 220 comprises a generally flexible tubular member 225 and with a hemostatic valve 227 at a proximal end thereof. The insertion sheath 220 includes a fold 224 disposed at a first or distal end 222 thereof. The fold 224 is shown more clearly in FIGS. 3-4. The fold 224 acts as a one-way valve to the anchor 108. As shown in FIG. 2A-2B and 3, the fold 224 is a plastic deformation in a portion of the insertion sheath 220 that elastically flexes as the anchor 108 is pushed out through the first end 222 of the insertion sheath 220. However, as the anchor 108 passes though and out of the first end 222 of the insertion sheath 220 as shown in FIG. 5, the fold 224 attempts to spring back to its original deformed position and a biased tip 226 of the fold 224 engages the nest 114. As relative movement between the carrier tube 102 and the insertion sheath 220 continues, the biased tip 226 traverses the contour 128 of the carrier tube nest 114 in a proximal direction.

Typically, after the anchor 108 passes through the first end 222 of the insertion sheath 220 and enters an artery 730 (FIG. 7), the puncture closure device 100 is pulled in a proximal direction with respect to the insertion sheath 220. The biased tip 226 of the fold 224 again follows the contour 128 and usually slides distally between the anchor 108 and the nest 114, causing the anchor to rotate as shown in FIG. 6. Accordingly, if all goes well, the anchor 108 is deployed within the artery as shown in FIG. 7 and does not reenter the insertion sheath 220.

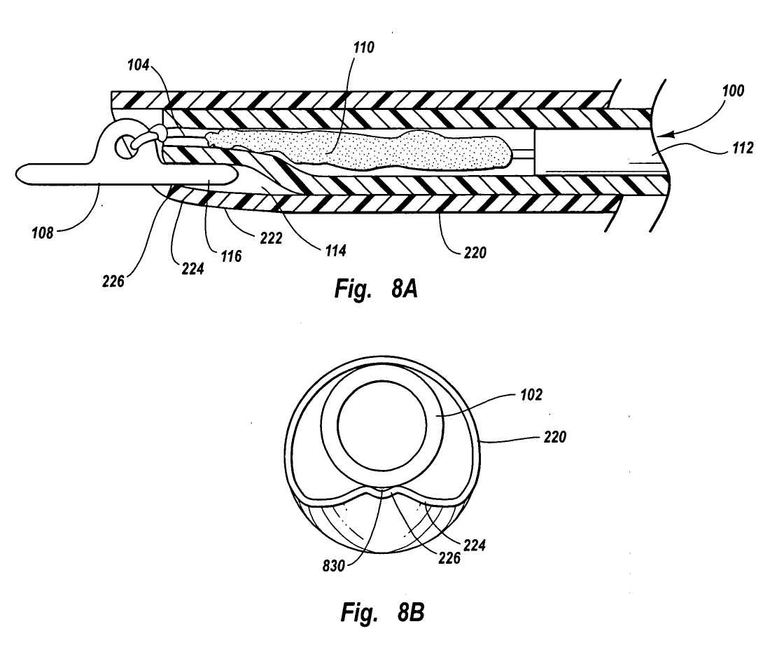

However, because the end 116 of the anchor 108 normally bears directly against the nest 114, sometimes the biased tip 226 of the fold 224 slides over the anchor 108 as shown in FIG. 8A when the closure device 100 is pulled proximally with respect to the insertion sheath 220, instead of sliding between the end 116 and nest 114. Thus, rather than deploying properly within the artery, the anchor 108 is sometimes reinserted into the insertion sheath 220, and the puncture closure device 100 fails.

One reason the anchor 108 sometimes slides back under the fold 224 is the tendency of the typical fold 224 to pucker as the closure device 100 is inserted through the fold 224. Referring to FIG. 8B, when the closure device 100 or other instrument passes through the insertion sheath 220, it is possible for the tip 226 of the conventional fold 224 to pucker and create a gap 830 between the carrier tube 102 and the insertion sheath 220. The gap 830 is sometimes wide enough to allow reentry of the anchor end 116 (FIG. 1B), and the fold thus no longer acts as a one-way valve.

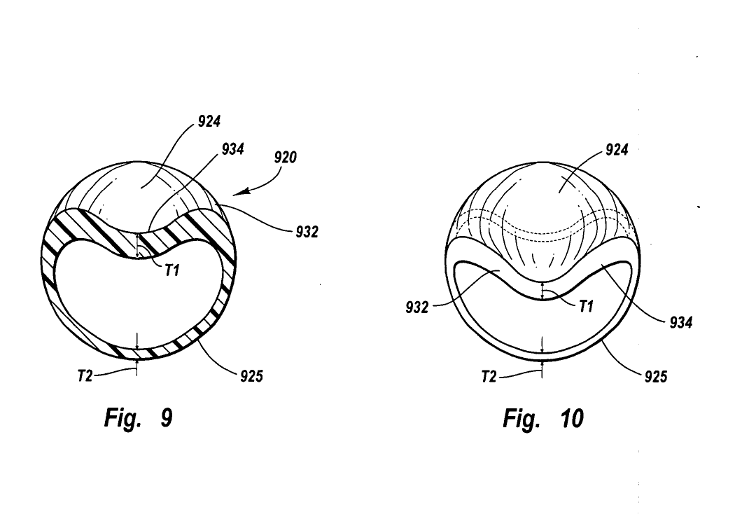

Therefore, according to some embodiments of the present invention, a tissue closure assembly includes a modified insertion sheath 920 as shown in FIG. 9. The modified insertion sheath 920 includes a flexible tubular member 925 (and in some embodiments a hemostatic valve 227 at a proximal end thereof as shown in FIG. 1A) and a tip portion 932. According to principles described herein, the tip portion 932 is rendered stiffer than the tubular member 925. As shown in FIG. 9, the tip portion 932 comprises a concave fold 924 with an edge section 934. According to the embodiment of FIG. 9, the concave fold 924 comprises no more than about half of a circumference of the insertion sheath 920.

Stiffness is a characteristic of the resistance of a material or object to deformations. Therefore, the stiffness of the tip portion 932 may be characterized by a stiffness coefficient k. The stiffness coefficient k is commonly used as an experimental value to characterize elastic and viscoelastic materials. The coefficient k is normally expressed by k=∂F/∂x, where F is the applied load and x is the relative displacement. Stiffness is not conservative and depends on geometry as well as the material. Accordingly, the stiffness of the tip portion 932 or a segment of the tip portion 932 may be increased in a number of ways. Some exemplary methods of stiffening the tip portion 932 are described and illustrated below. However, it will be understood by those of skill in the art having the benefit of this disclosure that many other stiffening methods may also be used, and that the methods and apparatus described and illustrated below are not an exhaustive set. The present invention contemplates any stiffening of the tip portion 932 or a sub-part of the tip portion 932 of an insertion sheath with respect to the generally flexible tubular member 925.

According to the embodiment of FIG. 9, the fold 924 of the tip portion 932 is stiffened by a greater wall thickness T1 than a wall thickness T2 of the tubular member 925. The greater wall thickness T1 may continue throughout the entire fold 924, the entire tip portion 932, or a sub-part of the fold 924. In addition, the greater wall thickness T2 may be variable or constant across the tip portion 932. An increase in the wall thickness T1 results in a higher stiffness coefficient for the fold 924 (or other segments of the tip portion 932) and therefore a reduced tendency to pucker when the closure device 100 is passed therethrough. A reduction in pucker tendency results from stiffening because instead of puckering, a stiffened fold 924 will tend to move as a single rigid unit as the closure device 100 passes therethrough.

Similarly, in some embodiments only the edge section 932 of the fold 924 is stiffened by the greater wall thickness T1 as shown in FIG. 10. Increasing the wall thickness of just the edge section 932 may sufficiently stiffen the fold 924 to prevent pucker.

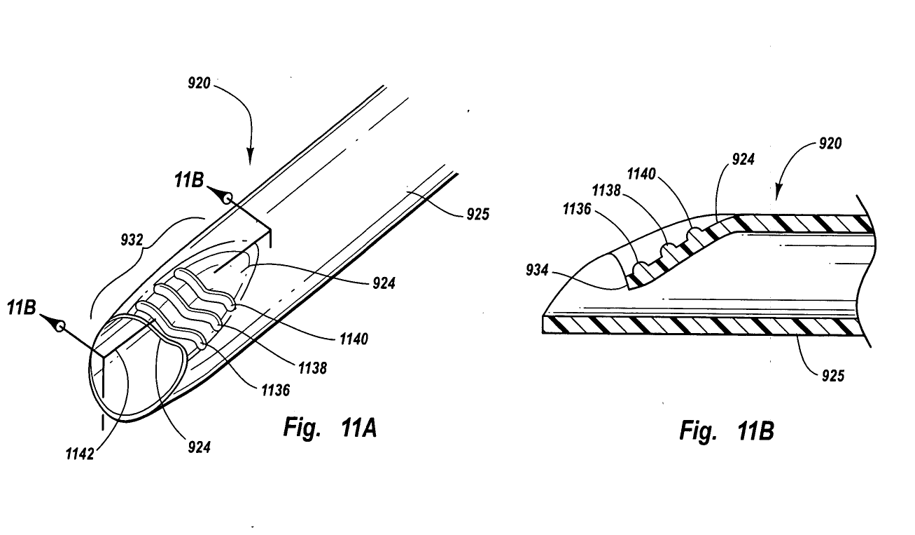

Turning next to FIGS. 11A-11B, another stiffening mechanism according to the present invention is illustrated. As shown in FIGS. 11A-11B, the tip portion 932 comprises at least one stiffening ridge 1136. The stiffening ridge is arranged substantially orthogonal to a longitudinal axis 1142 of the insertion sheath 920. The stiffening ridge 1136 is shown at the edge 934 of the fold 924 and reduces or eliminates the tendency of the fold 924 to pucker. There may also be additional stiffening ridges to increase the stiffness coefficient of the fold 924, such as the two additional stiffening ridges 1138, 1140 shown. The stiffening ridges 1136, 1138, 1140 are generally parallel to one another in the embodiment shown, but this is not necessarily so.

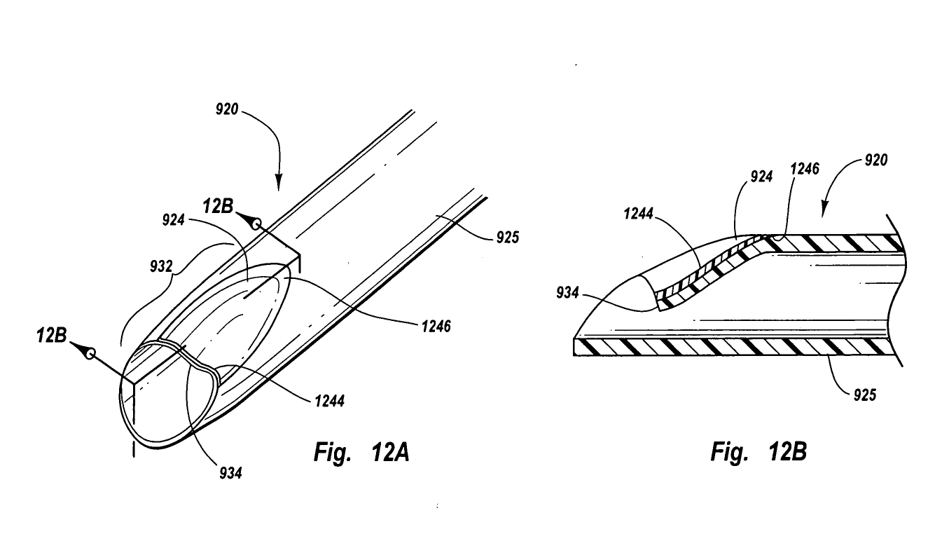

The tip portion 932 of the insertion sheath 920 may also be stiffened by the addition of a second layer of material 1244 as shown in FIGS. 12A-12B. According to the embodiment of FIGS. 12A-12B, the second layer 1244 coincides with, and therefore stiffens, the fold 924. The second layer 1244 may be of the same or a different material than the tubular member 925. The second layer 1244 may be of uniform or varying thickness. However, according to the embodiment shown, the second layer 1244 is thicker at the fold edge 934 where pucker is a problem than it is at a second end 1246 of the fold 924. According to some embodiments, the second layer 1244 is only added to the fold edge 934.

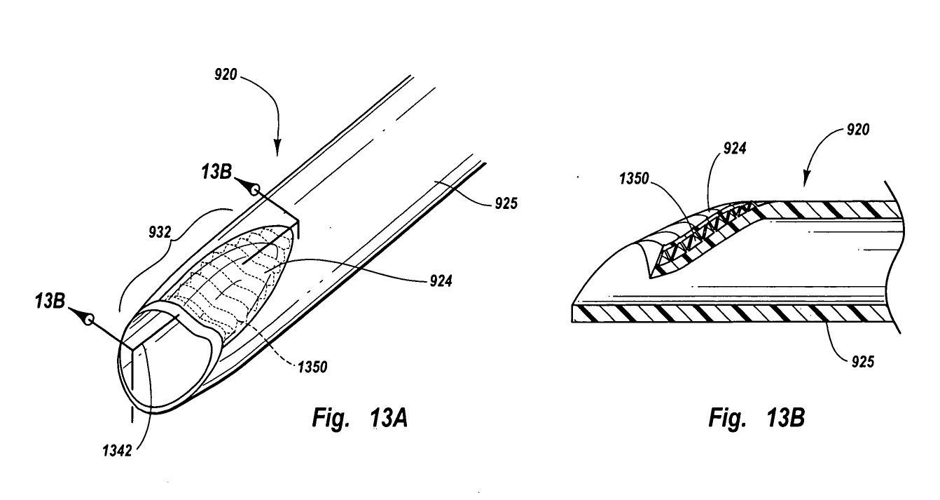

Referring next to FIGS. 13A-13B, another stiffening mechanism for the tip portion 932 of the insertion sheath 920 is shown according to the present invention. As shown in FIGS. 13A-13B, the tip portion 932 includes a corrugated section 1350 in the fold 924. According to FIGS. 13A-13B, the corrugated section is disposed transverse to a longitudinal axis 1342 of the insertion sheath 920, stiffening the fold 924 against pucker when the puncture closure device 100 (FIG. 1A) is inserted therethrough.

The various embodiments of the insertion sheath 920 shown and described above may be made by any of a number of ways. For example, the insertion sheath 920 may be made by providing the flexible tubular member 925, tapering the end portion 932 of the flexible tubular member for ease of insertion into a percutaneous incision, folding a section of the end portion 932 into a concave depression or fold 924, and stiffening at least a portion of the fold 924. The tapering, folding, and stiffening may each be accomplished by inserting the flexible tubular member 925 into one or more heated dies that reform the end portion 932. Reforming the flexible tubular member 925 to any of the configurations described above, or others, stiffen the end portion 932 and reduce the possibility of anchor shuttle.

The various modifications to the insertion sheath 920 may be implemented with any tissue puncture closure assembly, such as a tissue puncture closure assembly 1400 shown in FIG. 14. The tissue puncture closure assembly 1400 includes the closure device 100 for partial insertion into and sealing of an internal tissue wall puncture 1452. The closure device 100 is shown inserted through the insertion sheath 920, which has the stiffened tip portion 932. The stiffened tip portion 932 may be stiffened according to any of the embodiments described above or others.

An operator may seal the internal tissue wall puncture 1452 by inserting the tissue puncture closure device 100 through the insertion sheath 920 and into a percutaneous incision 1454. The anchor 108 is inserted through the puncture 1452 and into a lumen 1456. The anchor may be deployed in part by pulling the closure device 100 proximally back through the insertion sheath 920. The tip portion 932 of the flexible tube 925 acts as a one-way valve and forces the anchor 108 to rotate rather than allowing it to reinsert itself into the insertion sheath 920. Further, because the tip portion 932 is stiffened to reduce the occurrence of pucker, the chance of a reinsertion of the anchor 108 into the insertion sheath 920 is also reduced. The closure device 100 and insertion sheath 920 are then withdrawn from the percutaneous incision 1454 together, exposing the suture 104 and the tamping tube 112. The tamping tube 112 is then used to tamp the collagen sponge 110 or other sealing plug toward the anchor 108, such that the anchor 108 and the collagen sponge 110 sandwich and seal the puncture 1452. The suture 104 is then cut, leaving the anchor 108 and the collagen sponge 110 at the puncture 1452 site.

The words “including” and “having,” as used in the specification, including the claims, have the same meaning as the word “comprising.”

While the invention has been particularly shown and described with reference to embodiments thereof, it will be understood by those skilled in the art that various other changes in the form and details may be made without departing from the scope of the invention.

1. A tissue puncture closure assembly, comprising:
a tissue puncture closure device having a distal and a proximal end;
a vascular insertion sheath having a distal and a proximal end;
wherein the distal end of the insertion sheath comprises a tip portion that is stiffer than insertion sheath portions adjacent to the tip portion.

2. A tissue puncture closure assembly according to claim 1 wherein the tip portion comprises a concave fold.

3. A tissue puncture closure assembly according to claim 2 wherein the concave fold comprises no more than half of a circumference of the insertion sheath.

4. A tissue puncture closure assembly according to claim 1 wherein the stiffer tip portion comprises a wall thickness greater than a wall thickness of the insertion sheath adjacent to the tip portion.

5. A tissue puncture closure assembly according to claim 1 wherein the stiffer tip portion comprises a second layer of material.

6. A tissue puncture closure assembly according to claim 5 wherein the second layer of material is disposed substantially along a concave fold of the stiffer tip portion.

7. A tissue puncture closure assembly according to claim 5 wherein the second layer of material is disposed only along an edge of the stiffer tip portion.

8. A tissue puncture closure assembly according to claim 1 wherein the stiffer tip portion comprises at least one stiffening ridge.

9. A tissue puncture closure assembly according to claim 8 wherein the stiffer tip portion comprises at least two stiffening ridges.

10. A tissue puncture closure assembly according to claim 8 wherein the at least one stiffening ridge is arranged substantially orthogonal to a longitudinal axis of the insertion sheath.

11. A tissue puncture closure assembly according to claim 1 wherein the stiffer tip portion comprises a corrugated section.

12. A tissue puncture closure assembly according to claim 12 wherein the corrugated section is disposed transverse to a longitudinal axis of the insertion sheath.

13. A tissue puncture closure assembly according to claim 1 wherein the closure device comprises:
a filament extending from the proximal end of the closure device to the distal end of the closure device;
an anchor for insertion through a tissue wall puncture attached to the filament at the distal end of the closure device;
a sealing plug slidingly disposed about the filament at the distal end of the closure device.

14. A vascular insertion sheath, comprising:
a flexible tubular member having a longitudinal axis, a distal end, and a proximal end;
a hemostatic valve coupled to the proximal end of the tubular member;
a fold at the distal end of the tubular member, the fold comprising a higher stiffness coefficient than the tubular member.

15. A vascular insertion sheath according to claim 14, further comprising a layer of material over the fold to provide the higher stiffness coefficient.

16. A vascular insertion sheath according to claim 15 wherein the layer of material is placed only at an edge of the fold.

17. A vascular insertion sheath according to claim 14 wherein at least a portion of the fold comprises a thicker wall than the flexible tubular member.

18. A vascular insertion sheath according to claim 17 wherein only an edge of the fold comprises a thicker wall than the flexible tubular member.

19. A vascular insertion sheath according to claim 14, further comprising at least one stiffening ridge across the fold transverse to the longitudinal axis.

20. A vascular insertion sheath according to claim 14 wherein the fold is corrugated.

21. A vascular insertion sheath according to claim 20 wherein the corrugated fold is corrugated in a direction transverse to the longitudinal axis.

22. A method of reducing anchor shuttle in a tissue puncture closure assembly, comprising stiffening a tip portion of an insertion sheath receptive of a tissue puncture closure device.

23. A method of reducing anchor shuttle in a tissue puncture closure assembly according to claim 22 wherein the stiffening further comprises increasing a wall thickness of the tip portion of the insertion sheath.

24. A method of reducing anchor shuttle in a tissue puncture closure assembly according to claim 23, further comprising increasing the wall thickness of only a folded section of the tip portion.

25. A method of reducing anchor shuttle in a tissue puncture closure assembly according to claim 23, further comprising increasing the wall thickness of only an edge of the tip portion.

26. A method of reducing anchor shuttle in a tissue puncture closure assembly according to claim 22 wherein the stiffening further comprises applying a layer of material to the tip portion of the insertion sheath.

27. A method of reducing anchor shuttle in a tissue puncture closure assembly according to claim 25 wherein the stiffening further comprises applying a layer of material only to a folded section of the tip portion of the insertion sheath.

28. A method of reducing anchor shuttle in a tissue puncture closure assembly according to claim 22 wherein the stiffening further comprises corrugating the tip portion of the insertion sheath.

29. A method of reducing anchor shuttle in a tissue puncture closure assembly according to claim 28 wherein the corrugating is done in a direction transverse to a longitudinal axis of the insertion sheath.

30. A method of reducing anchor shuttle in a tissue puncture closure assembly according to claim 28 wherein the corrugating is done only in a folded section of the tip portion.

31. A method of reducing anchor shuttle in a tissue puncture closure assembly according to claim 22 wherein the stiffening further comprises adding a ridge across the tip portion in a direction transverse to a longitudinal axis of the insertion sheath.

32. A method of reducing anchor shuttle in a tissue puncture closure assembly according to claim 31 wherein the stiffening comprises adding a ridge only across a folded section of the tip portion.

33. A method of making a vascular insertion sheath, comprising:
providing a flexible tubular member receptive of a puncture closure device;
tapering an end portion of the flexible tubular member;
folding a section of the end portion into a concave depression;
stiffening at least a portion of the concave depression.

34. A method of making a vascular insertion sheath according to claim 33 wherein the tapering comprises inserting the flexible tubular member into a heated die and reforming the end portion.

35. A method of making a vascular insertion sheath according to claim 33 wherein the folding further comprises inserting the flexible tubular member into a heated die and reforming the end portion.

36. A method of making a vascular insertion sheath according to claim 33 wherein the stiffening further comprises inserting the flexible tubular member into a heated die and reforming at least part of the end portion into a thicker wall or a corrugated section.

37. A method of making a vascular insertion sheath according to claim 36 wherein only a folded section or an edge of the folded section is reformed into the thicker wall or corrugated section.

38. A method of making a vascular insertion sheath according to claim 33 wherein the stiffening further comprises applying a layer of material to at least part of the end portion.

39. A method of making a vascular insertion sheath according to claim 33 wherein the stiffening further comprises adding a ridge across the end portion in a direction transverse to a longitudinal axis of the flexible tubular member.

40. A tissue puncture closure assembly, comprising:
a closure device for partial insertion into and sealing of an internal tissue wall puncture, the closure device comprising:
a carrier tube having an anchor nest at a distal end;
a filament extending through the carrier tube;
an anchor attached to the filament at the distal end of the carrier tube and seated in the anchor nest;
an insertion sheath receptive of the carrier tube of the closure device, the insertion sheath comprising:
a flexible tubular member having a longitudinal axis, a distal end, and a proximal end;
a hemostatic valve coupled to the proximal end of the tubular member;
a fold at the distal end of the tubular member, the fold comprising a higher stiffness coefficient than the tubular member.

41. A method of sealing a tissue puncture in an internal tissue wall accessible through a percutaneous incision, comprising:
providing a tissue puncture closure device comprising a carrier tube with a filament extending therethrough, the filament connected at a distal end of the carrier tube to an anchor, the anchor seated in a nest disposed in the carrier tube, the filament also connected to a sealing plug located proximal of the anchor for disposition and anchoring about the tissue puncture;
inserting the tissue puncture closure device through an insertion sheath having a stiffened tip portion into the percutaneous incision;
deploying the anchor into the tissue puncture;
withdrawing the closure device from the percutaneous incision;
tamping the sealing plug toward the anchor.
